(19) United States
(12) Patent Application Publication
(10) Pub. No.: US 2016/0296124 A1
(43) Pub. Date: Oct. 13, 2016

(54) Wearable Wireless Multisensor Health Monitor with Head Photoplethysmograph

(71) Applicant: Venture Gain, LLC, Naperville, IL (US)

(72) Inventors: Stephan W. Wegerich, Geneva, IL (US); Robert Matthew Pipke, Oak Park, IL (US); Thaddeus Meizelis, Plainfield, IL (US)

(73) Assignee: Venture Gain L.L.C.

(21) Appl. No.: 15/186,296

(22) Filed: Jun. 17, 2016

Related U.S. Application Data
(63) Continuation of application No. 13/840,179, filed on Mar. 15, 2013, now abandoned.

(51) Int. Cl. A61B 5/0205; A61B 5/00; A61B 5/024; A61B 5/026; A61B 5/053; A61B 5/01; A61B 5/021; A61B 5/0408; A61B 5/0245

(52) U.S. Cl. CPC A61B 5/0205; A61B 5/04085; A61B 5/6823; A61B 5/6831; A61B 5/6815; A61B 5/6814; A61B 5/6833; A61B 5/0024; A61B 5/0006; A61B 5/02416; A61B 5/0245; A61B 5/053; A61B 5/01; A61B 5/021; A61B 5/0261

(57) ABSTRACT

Ambulatory monitoring of human health is provided by a multi-component multi-sensor wireless wearable biosignal acquisition system comprising a torso device and a peripheral device communicating wirelessly, and a mobile phone for receiving collected data and uploading it over cellular network or WiFi to a remote computer for multivariate analysis. Biosignals include EKG and PPG, from which a determination of pulse transit time can be made.

### STATEMENT REGARDING FEDERALLY SPONSORED RESEARCH OR DEVELOPMENT

This invention was made with Government support under contract order number VA118-11-P-0031 awarded by the Department of Veterans Affairs. The Government has certain rights in this invention.

### BACKGROUND OF THE INVENTION

1. Field of the Invention

The present invention relates generally to the field of human health monitoring, and more particularly to wearable wireless devices for collecting continuous measurements of biological parameters to provide an assessment of human health and wellness.

2. Brief Description of the Related Art

Current commercially available equipment for medical monitoring outside of the acute care setting provides woefully inadequate visibility into patient health because of the paucity of data collected. Typically, only one variable is collected, without reference to other parameters. Moreover, data is typically collected at very low data rates, for example just once per day. In many cases, the capture of data occurs only when the patient takes the trouble to use the equipment, as for example in the case of an inflatable blood pressure cuff for a blood pressure measurement unit, or a weight scale. While devices for capturing electrocardiograms (ECG or EKG) at high sampling rates have been well known for decades, such as the Holtor monitor and Event monitor as well as certain implanted sensing pacemakers and implantable cardioverter defibrillators (ICD), this information alone is insufficient to characterize the overall health of the patient. In any case, these devices are often used only sporadically, and may only capture data for short intervals. Continuous capture, especially in an ambulatory circumstance, of multivariate data characterizing the physiology of the patient has been beyond the reach of commercially viable equipment and devices.

As people live longer, and hence live with dangerous chronic diseases, there is a growing need for monitoring these patients at home in their daily lives, and to provide medical clinicians visibility into patient status so that health can be optimally maintained, exacerbations of these conditions can be ameliorated early and episodic hospitalization can be avoided. Such an approach to health care for an ever larger population of patients living outside a critical care setting with a chronic condition that can deteriorate unexpectedly and rapidly at any time holds great potential to reduce costs across the health care system and improve patient compliance with medication, diet and exercise, and improve outcomes. This kind of real-time visibility into health status could also be advantageously incorporated into a patient self-treatment feedback loop, allowing patients to better manage their health.

In order to provide adequate surveillance for this new approach to patient care, as well as health and wellness optimization in healthy people, better physiological telemetry is needed. Sensors and devices need to be smaller, lighter and less stigmatizing, so that people are willing to regularly use them. Physiological telemetry also needs to cover more time; something closer to continuous data is needed. The reason is that early indicators of health change or deterioration are obfuscated by normal daily variation of human physiology responsive to normal metabolic, activity and diurnal demands. Measurements of physiological parameters on a “spot-check” basis can be rather meaningless except with respect to the coarsest of changes, given the normal background variation present in these parameters. For example, a spot check measurement of blood pressure in the at-home environment can easily exhibit wide variation depending on whether or not the patient rests calmly before taking the reading. Outside of large magnitude changes, this reading may not contain much information by itself in isolation from other vital signs. Moreover, a time series of such readings is likely to be only a lagging indicator of initial health deterioration rather than a leading indicator on which clinicians could proactively intervene. Early, incipient signs of health changes manifesting in subtle changes to blood pressure can only be ferreted out in the context of analyzing multiple vital signs together and collecting continuous data. Therefore, what is needed is a mobile multi-sensor device capable of collecting near-continuous or continuous data, which is easily worn by the patient.

In many chronic disease exacerbations, health changes are most immediately seen in parameters that characterize the cardio-pulmonary control system of the human body, since this is the system most critically targeting homeostasis. Such parameters as heart rate, respiration rate, blood pressure and pulse oximetry are typically captured in the acute care setting as leading indicators of acute health degradation. While these parameters are easily measured in the controlled environment of the hospital, where the patient is likely sedated and supine, they are more difficult to obtain in the home ambulatory environment, where daily activity can introduce problematic motion artifact and where sensors are not as easily attached to the patient. Respiration rate is very difficult to measure in the ambulatory environment in which it is unlikely the patient will tolerate a device covering the mouth or nostrils to measure flow, and where other indicators of respiration are confounded by motion artifact. Pulse oximetry is notoriously difficult with changing ambient light, which introduces interference with light signals of the pulse oximeter, and moreover with bodily motion, which actually interferes with the blood flow impulse that is the basis of the calculation of oxygen saturation (SpO2). Blood pressure is perhaps the most difficult of all, since conventional methods involve holding still while a pressure cuff is inflated around the arm or wrist, while sitting in a calm and repeatable posture. What is needed is a way to measure parameters like these that characterize the cardiopulmonary control system of the human body in a continuous fashion in an ambulatory environment.

### SUMMARY OF THE INVENTION

A wearable wireless system for acquisition of continuous physiological parameters is disclosed, for use in monitoring the health and wellness of a human. The system comprises one or more devices, each with a microprocessor for acquisition of data from sensors connected thereto. The devices communicate wirelessly with one another. Sensor data is aggregated across devices by transmission to a mobile (cellular) phone, which then is capable of relaying the data to a remote computer for analysis via cellular network, local wireless network such as WiFi, or other telecommunications method by which a phone can send information.

The system can be used for remote patient monitoring in the ambulatory environment of the home and work, to facilitate early detection of incipient health problems for early intervention by medical clinicians in order to prevent subsequent exacerbation and hospitalization. This allows patients with chronic diseases or medical conditions prone to deterioration, to live high quality lives away from an acute care setting while still being effectively monitored by medical staff. Data collected by the system of the current invention is uploaded to a remote computer where it is analyzed for indications that patient health is changing or deteriorating; this information is then presented to medical clinicians, typically through a computer interface such as a web browser or via notification on a mobile or portable communications device, who can contact the patient to encourage medication compliance, change medications, change medication dosage, encourage dietary compliance, invite the patient to come to a non-acute care setting for testing, and take other low-cost steps to help the patient ameliorate further deterioration and hospitalization.

The system can also be used for medical monitoring in an acute care setting. Because the system is wearable, it can be easily moved with the patient, while maintaining constant data collection. Data can be transmitted via hospital WiFi network, so that patient physiological parameters are continuously monitored even as the patient is moved from one setting to another (e.g., hospital room to radiology).

In one embodiment, the system comprises a torso device for sensing parameters from the human torso, and a peripheral device for sensing parameters from the head (or limb) of the human. The torso device is worn under clothing. Both units are worn together, and can be worn for many hours to provide continuous data. The devices communicate data wirelessly, and the data is transmitted and aggregated on a mobile phone carried by the human. The mobile phone uploads data periodically via a cellular phone network or WiFi to one or more remote computers, such as an analytics data center, for analysis. The torso device measures one or more of: electrical activity of the heart in the form of an electrocardiogram; trans-thoracic bioimpedance as a measure of respiratory activity; 3-axis accelerometry as a measure of posture and activity; and temperature. The torso device is physically connected to four or more electrodes placed on the skin of the torso, two of which are used to inject the bioimpedance current. The torso device may be worn in the form of a belt around the chest, which provides skin contact for the electrodes on the inside of the belt. It may also be worn in a form whereby it is adhesively attached to the skin, as are the electrodes. The peripheral device measures one or more of: volumetric pulsatile blood flow in the capillary bed of the tissue in the form of a photoplethysmogram; oximetry by means of two-color differential absorption from the photoplethysmogram; motion and orientation by 3-axis accelerometry; skin temperature; and ambient temperature. The peripheral device may be worn against the forehead, held in place by a headband or held in place adhesively. Alternatively, the peripheral device may be worn against the skin over the mastoid process bone behind the ear, held in place adhesively. The data from the two devices is used in a synchronized fashion in order to determine joint physiological measurements, which includes a measure of the transit time of a heart beat pulse wave in the arterial network, as measured from the initiation of the heartbeat. Data is transmitted wirelessly from the peripheral device on the head to the torso device by a paired radio link. The torso device combines the data from the head peripheral device with its own data, and relays this via another radio link (typically Bluetooth) to the mobile phone.

The mobile phone receives the data from the torso device in packets at a configurable rate, which may be virtually continuous, or may be in one-second bursts for example, in order to conserve battery life by turning off the radio between transmissions. The mobile phone has a graphical interface that can display the physiological biosignals comprised of the data sent by the torso device, such as the electrocardiogram (ECG or EKG), the photoplethysmogram (PPG), the bioimpedance voltage, and so on. The mobile phone also is capable of processing these biosignals to derive vital sign “features” from them, such as determining heart rate from the EKG. The mobile phone is configurable to upload derived features and/or raw biosignals to one or more remote computers for analysis.

Uploaded features are advantageously analyzed using a multivariate residual-based physiology modeling approach. A multivariate kernel-based model is developed based on normal physiology (preferably personalized to the patient who is wearing the wearable monitor of the invention). This model then makes estimates of the expected values for the vital sign features in response to being presented with monitored values of those features, either uploaded from the mobile phone, or derived on the remote computer(s) using the raw biosignals uploaded from the mobile phone. These estimates are compared to the monitored values of the features, and discrepancies (also known as residuals) between the expected values of the estimates and the monitored values are indicated as signs of health deviation from normal physiological behavior. Such deviations are further analyzed to assess the degree of health abnormality, which can be conveyed to a medical clinician as early warning of patient health degradation, and the clinician can proactively contact the patient to intervene and avoid hospitalization, or in the case of a patient already convalescing in an acute care setting, medical staff can triage the patient on a prioritized basis.

In one embodiment, the peripheral device is worn behind the ear of the human on the skin above the mastoid process bone. The ear device projects the output of at least one light emitting diode (LED) toward the skin, and a photodetector in the ear device placed close by detects light returned from the tissue of the skin, subject to absorption by the tissue and by the blood in the capillary bed of the tissue, providing a time-varying PPG signal indicative of pulsatile blood flow. In order to properly place both the LED and the photodetector in proximity to the skin to obtain a clear signal, the ear device is cupped to fit the curvature of the mastoid process. Further, the ear device is held in place by a double-sided adhesive around the perimeter of the PPG LED and photodetector. The LED and photodetector moreover extend above the cupped surface and into the skin a sufficient distance such that substantially all light from the LED can travel to the photodetector only by passing through the tissue; however the distance of extension is small enough that pressure exerted by the LED and photodetector against the skin does not significantly occlude blood flow in the capillary bed of the tissue.

The peripheral device synchronizes its data transfer with the torso device to assure that a constant offset between the PPG signal from the peripheral device and the EKG signal obtained by the torso device remains accurate to within a preselected tolerance. A time difference is determined between repeating landmarks of each of these biosignals as an indicator of the pulse transit time of pulsatile blood flow, which in turn provides a continuous indication of arterial compliance and blood pressure.

Another embodiment of the present invention is a system for monitoring human health comprising a wearable torso device disposed to continuously measure at least an electrocardiogram signal from at least two electrodes on the torso; a head-worn device in wireless connectivity with the torso device, having at least one light source disposed to illuminate the capillary bed below the skin at a location on the head and having a light sensitive element for quantitatively measuring light from said light source that has passed through the capillary bed, thereby providing a photoplethysmogram signal that is communicated to the torso device wirelessly; where said torso device and said head-worn device have a mechanism implemented in software for synchronizing the electrocardiogram signal with the photoplethysmogram signal for an accurate determination of a pulse transit time.

### BRIEF DESCRIPTION OF THE DRAWINGS

The novel features believed characteristic of the invention are set forth in the appended claims. The invention itself, however, as well as the preferred mode of use, further objectives and advantages thereof, is best understood by reference to the following detailed description of the embodiments in conjunction with the accompanying drawings, wherein:

FIG. 1 shows a human wearing an embodiment of the multi-sensor system of the present invention;

FIGS. 2A and 2B show two alternative configurations for wearing the torso device according to the invention;

FIG. 3 shows an embodiment of the peripheral device of the present invention suitable for wearing on the forehead;

FIG. 4 shows an embodiment of the peripheral device of the present invention suitable for wearing behind the ear;

FIG. 5 shows another embodiment of the peripheral device of the present invention suitable for wearing behind the ear;

FIG. 6 is a cross sectional view of the PPG sensor of FIG. 4;

FIG. 7 is a cross sectional view of the PPG sensor of FIG. 5;

FIG. 8 shows an embodiment of the torso device of the present invention;

FIGS. 9A and 9B show an embodiment of a chest belt harness for the torso device; and

FIG. 10 shows an embodiment of an adhesive harness for the torso device;

### DETAILED DESCRIPTION OF THE PREFERRED EMBODIMENTS

The remote health monitoring system of the present invention is effective for remote patient health monitoring in the at-home, ambulatory environment. It is intended to be worn comfortably and innocuously by a patient under clothing and in the course of normal, daily living without significant hindrance to mobility or activity. It is designed to be worn by the patient for many hours each day, every day. It is designed with sufficiently lightweight batteries such that it should be recharged on a daily basis. It continuously monitors multiple biosignals noninvasively, and wirelessly transmits data to a remote server, to enable monitoring, analysis and proactive alerting of incipient health issues for the patient. It may advantageously be used in monitoring chronically ill patients, such as heart failure patients, who can experience unexpected exacerbations of their condition leading to the need for emergency acute medical treatment.

The monitoring apparatus comprises at least three components. A first component is a device mounted on the torso (“torso device”), under clothing, and connected to at least four electrodes in contact with the skin of the torso. A second component is a device worn at the periphery of the body (“peripheral device”), especially on the head, which primarily serves to acquire photoplethysmogram (PPG) signals. A third component is a mobile phone that receives data from the devices and uploads data to remote computers for multivariate analysis. Additional peripheral devices can be employed, for example at the wrist, hand or ankle. In a preferred embodiment, the peripheral device is mounted on the forehead or over the mastoid process behind the ear.

These components form a wireless multivariable sensor ensemble for continuous or semi-continuous biosignal collection for a determination of human health status. Biosignals that are collected include electrocardiogram (ECG or EKG) from two or more electrodes on the torso; trans-thoracic bioimpedance (BIOZ) voltage collected from two electrodes on the torso; 3-axis accelerometer signals (ACT) from a 3-axis accelerometer semiconductor device mounted in relation to the torso device as well as from a second such accelerometer mounted in relation to the peripheral device; a photoplethysmogram signal (PPG) for at least one light wavelength, and preferably at least two different wavelengths, obtained from the tissue at the location of the peripheral device; skin temperature obtained from one or both of the skin under the torso device or peripheral device; and air temperature preferably obtained from an outward-facing sensor on the peripheral device. Preferably, biosignals are collected at sampling rates of at least approximately 250 Hz to sufficiently characterize important aspects and landmarks of the biosignal waveforms having physiological significance, while balancing with power requirements of higher sampling rates. However, accelerometer signals can be acquired at about 100 Hz.

In an embodiment of the invention, the biosignals can be acquired as described below:
Electrocardiograph. Two leads are connected to the torso, one at approximately the position of conventional EKG lead V5 and the other in a mirror position on the opposite side of the rib cage. EKG is captured at a sampling rate of 250 Hz, using an ADS1294R chip from Texas Instruments.
Photoplethysmograph. PPG is captured using conventional two-color (red and infra-red) reflectance pulse oximetry. Each color is switched on/off at 250 Hz and shone into the skin of the patient at either the forehead or at the mastoid process behind the ear. An adjacent photodetector picks up reflected light having passed through the capillary bed.
Bioimpedance. The principle of bioimpedance is to measure the opposition to current flow in tissue when a high-frequency current is injected. Fluid filled compartments of the body conduct the current better, while air space, lipids and most carbon-chain-based tissue structures have low or no conductivity. A high frequency (64 kHz) micro-current (˜29 μA) is injected between two electrodes placed at the left and right lower rib cage, lateral to each of the EKG electrodes and spaced between ¼ and ½ inch from them. The bioimpedance electrodes are located further toward the side of the body than the EKG electrodes. The EKG electrodes detect the resultant voltage, which is high-pass filtered and compared with the injected excitation current to yield the impedance across the torso from side to side. This signal fluctuates both with arterial blood flow and with respiratory activity. Body movement also results in redistribution of conductive pathways (primarily fluid) amongst tissues and creates substantial motion artifact.
Acceleration. A three-axis accelerometer is mounted on the printed circuit board of the torso module. It is sampled at about 100 Hz to provide voltage signals characterizing both motion acceleration and orientation in the earth's gravitational field for the X-, Y- and Z-axes of the module. Hence it is important that the torso module be attached or positioned tightly on the body so that its orientation mirrors the orientation of the patient. Three signals are obtained. These signals can be combined to generate a single scalar gross activity, and the 3-dimensional orientation.
Temperature. A 10 Kohm 2-wire passive device (thermistor) coupled to thermally conductive pads against the skin or open to ambient air is sampled at 1 Hz.

Both the torso device and the peripheral device comprise a microprocessor, firmware memory for storing program code, internal memory for storing and buffering data, analog-to-digital converters for each channel of sensor data collected, a radio for paired bi-directional wireless transmission and reception of data and commands, and a power source. In a preferred embodiment, data collected by the peripheral device is wirelessly transmitted to the torso device, where it is combined by the microprocessor of the torso device with data collected from the torso device sensor channels. The radios used between the peripheral device and the torso device can be selected from a variety of bi-directional point-to-point pairing radios employing frequencies in the industrial, scientific and medical (ISM) radio bands, preferably around 915 MHz, and capable of emulating a serial port protocol. The torso device has a second radio, by way of example a Bluetooth standard radio, for communicating data and commands with a mobile phone. Preferably, the radio link between the torso device and the mobile phone employs the serial port profile (SPP) of the Bluetooth communication protocol standard. In this configuration, data is generally transmitted by the peripheral device to the torso device, where biosignals are synchronized in time, and then the torso device communicates the unified data to the mobile phone over a separate radio interface.

In another embodiment, the two devices and the mobile phone can form a multi-nodal network, for example using the Personal Area Network (PAN) profile of the Bluetooth communication protocol standard. In this event, the data collected by the peripheral device is transmitted directly to the mobile phone, where it is synchronized with the data transmitted from the torso device. An advantage of this configuration is that data can still be collected from the peripheral device even when the torso device fails. However, this configuration is not as energy efficient and may require larger batteries because of the use of the relatively energy-expensive Bluetooth radio standard at both body worn devices.

Extraction of vital sign features from biosignal data can be performed in the central processing unit (CPU) of the mobile phone, whereupon the mobile phone stores and forwards vital sign feature data to a remote computer for analysis when the mobile phone has data connectivity, preferably over the internet, via digital cellular transmission or via internet-connected WiFi access point. Alternatively, the mobile phone can upload the raw biosignal data to the remote computer, where vital sign feature extraction can be performed prior to multivariate analysis. The advantage of uploading raw biosignals is that they can be stored and reprocessed at a subsequent time with additional algorithms as they arise, whereas if features are calculated locally on the mobile phone and only the features are uploaded, there is no opportunity for reprocessing the biosignals. However, the advantage of uploading only features is that typically features require much less bandwidth than uploading raw biosignals, as can be understood from the discussion of feature extraction below. In a preferred embodiment, the mobile phone software is configurable to upload biosignal data, calculate and upload feature data, or to do both, depending on mobile phone processor capacity, local memory and cellular bandwidth.

Features extracted from the biosignals are generally at a much lower data rate, e.g., 2 Hz or less. Features can also be statistically summarized at regular intervals, such as once per minute or one per quarter minute. Features preferably extracted according to the invention are described as follows: Instant heart rate can be obtained by identifying the QRS complex of each heartbeat and using the time difference of successive QRS complexes. Instant heart rate can be time-stamped with the time of the QRS peak of one of the two beats involved in the time difference; can be sampled at 1 Hz by assigning the instant heart rate for the nearest QRS peak to the 1 Hz trigger; or can be averaged over a moving window to provide a different periodic ratefs. Heart rate variability can be computed from the variability exhibited in a time window of instant heart rates. Respiration rate can be obtained by inflexion point identification on trans-thoracic bioimpedance, which oscillates with thoracic expansion and contraction associated with breathing, and time-differencing the matching inflexion points of the signal. Alternatively, a spectral analysis can be performed over a time window (typically 15 seconds or more) of a low-bandpass filtered bioimpedance to identify power peaks within a physiologically plausible range of breathing frequencies, typically 8-40 breathes per minute, to identify the time window averaged respiratory rate. In yet another alternative, the oscillating envelop of ECG signal magnitude can be spectrally analyzed to identify respiratory rate. A measure of respiratory effort that can be associated with classic gas exchange parameters like Tidal Volume can be obtained from the magnitude of the dominant power peak in the spectral analysis of bioimpedance described above at the frequency determined for respiratory rate. General bodily activity can be calculated from the RMS amplitude of the three axes of accelerometer, preferably on the torso device. Differential temperature can be determined from the difference of skin temperature from either the torso device or the peripheral device with the ambient temperature from the peripheral device. Blood oxygenation, and particularly saturation of peripheral oxygen (SpO2), can be determined from the differential absorption of two wavelengths of light as determined from the amplitudes of the oscillatory components of transmitted PPG signals from red and infrared light emitting diode sources in the peripheral device. Pulse transit time can be determined as a function of the time difference between the QRS complex of the ECG signal from the torso device and the next inflexion point of the PPG signal from the peripheral device indicative of blood pulse arrival. A measure of diastolic relaxation rate can be determined from the inflexion point of the PPG signal associated with the a reversal from decreasing light transmission to increasing light transmission, as blood inflow to the capillary bed under surveillance begins to fall behind blood ebb, and the time interval between that inflexion point and the inflexion point associated with blood pulse arrival time, or the QRS complex of the ECG signal. A measure of pulse pressure index can be obtained by differencing that measure of diastolic relaxation with the pulse transit time. Posture can be obtained from the 3-axis accelerometer as a function of the three individual voltages representing the three axes as a map to known orientations in the gravitational field.

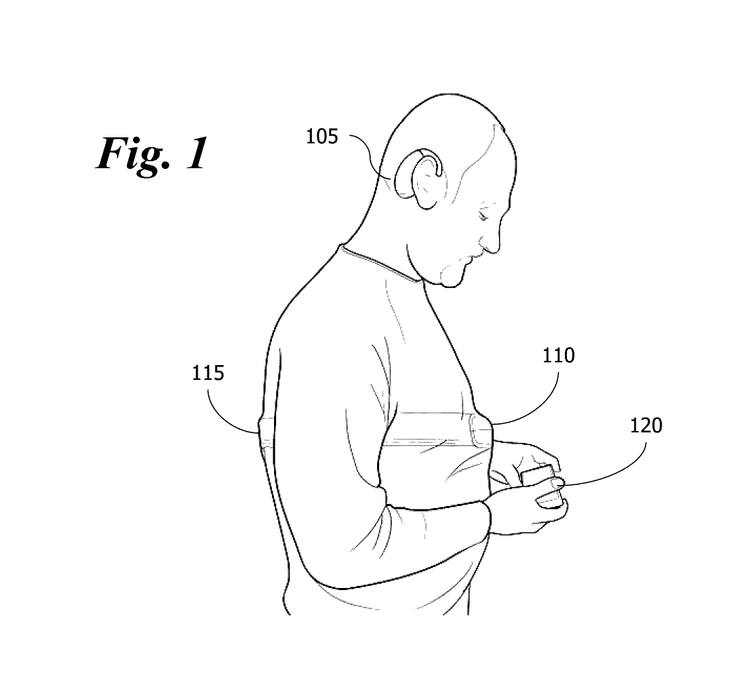

Turning to FIG. 1, one configuration for wearing the system of the present invention is shown on a human subject. In this configuration, the peripheral device takes a form 105 worn behind the ear over the mastoid process, directing the onboard light source down into the skin and tissue over that region, and similarly collecting the PPG signal with a photodetector similarly directed toward the skin. The PPG signal is thus a reflectance PPG, comprising light that has traveled into the tissue and scattered back out toward the photodetector. The peripheral device 105 communicates wirelessly with a torso device 110, worn in a chest belt 115 under clothing. The torso device 110 receives data from the peripheral device 105, combines that data with data collected by the torso device, and transmits the combined data to mobile phone 120. Raw biosignals or extracted features are uploaded by the mobile phone 120 via WiFi or cellular network.

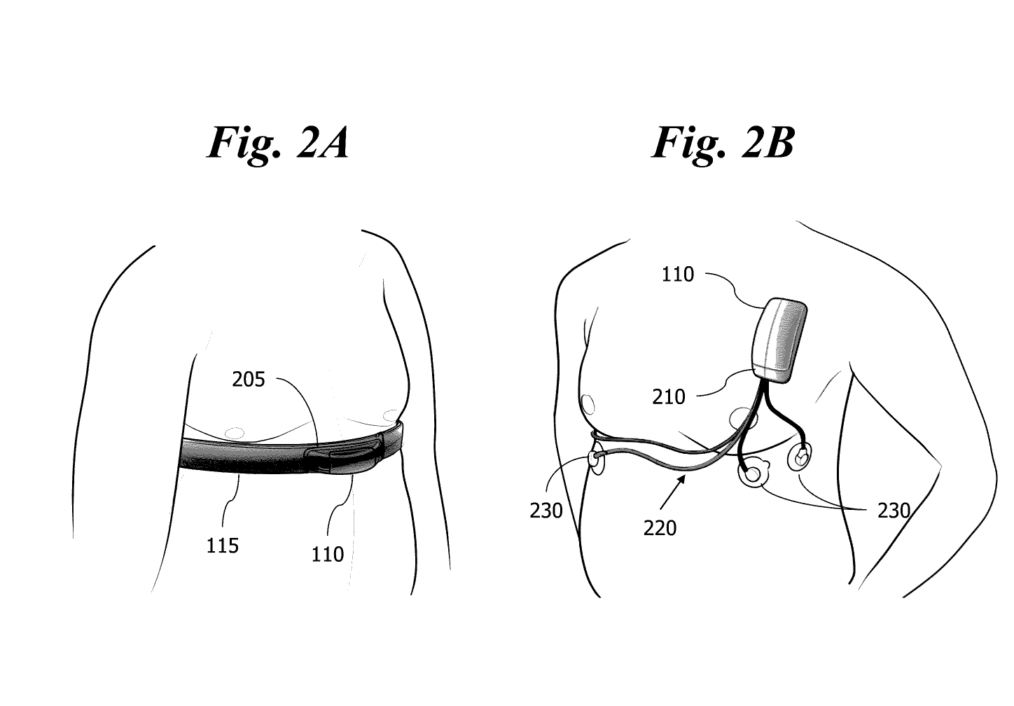

Turning to FIGS. 2A and 2B, two alternative configurations are shown for wearing the torso device 110. In FIG. 2A, a chest belt 115 is shown positioned approximately below the breast or nipple area, and above the diaphragm. The torso device 110 snaps into a receiving harness 205 that is mechanically attached to the chest belt 115, and provides electrical connectivity to at least four electrodes located on the inner surface of the chest belt and thereby held in contact with the skin. The electrodes can be conductive carbonized dry rubber electrodes such as those typical used in transcutaneous electrical nerve stimulation (TENS) applications. In FIG. 2B, the torso device 110 snaps into a receiving harness 210, which attaches adhesively to the skin of the upper chest. The harness 210 provides electrical connectivity to at least four electrical leads 220, which can be connected to disposable adhesive electrodes 230 attached directly to the skin. Common commercially available hydrogel disposable electrodes with standard button snap connectors are adequate for use in the invention. Electrode placement in FIG. 2B provides a good approximation for the intended location of the electrodes held in place by the chest belt 115 in FIG. 2A. Both harnesses can be designed to receive the same torso device, so that both modalities can be made available to a patient; each harness can connect electrically with the torso device by means of spring loaded pins that contact conductive pads on the surface of the torso device.

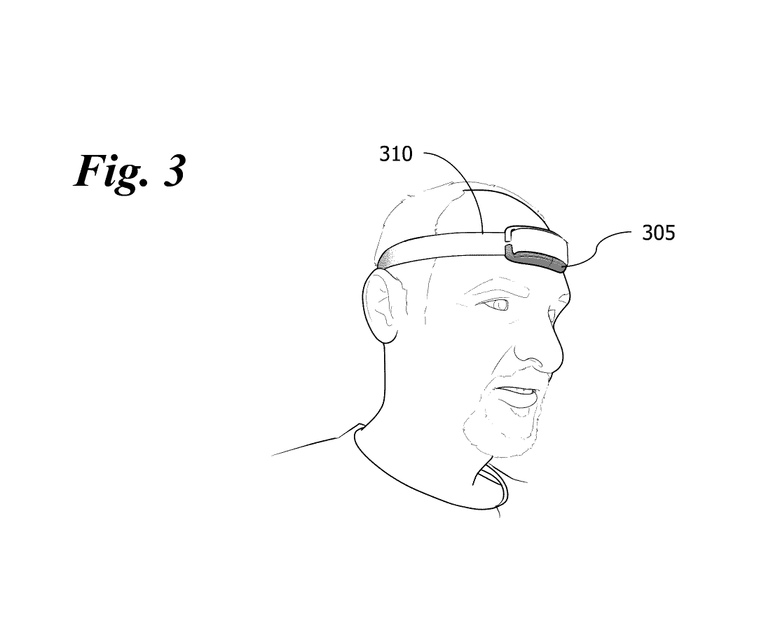

FIG. 3 shows how another embodiment of the peripheral device is worn on the forehead. Here, peripheral device 305 is held in place on the skin of the forehead by a headband 310. PPG-related light sources and photodetector are directed toward the skin of the forehead. A skin temperature sensor is also placed on the inner surface of peripheral device 305, typically comprising a thermistor connected to a thermally conductive contact plate. Peripheral device 305 comprises a curved enclosure to conform to the forehead curvature of the patient. The inner surface of the device may comprise flexible opaque foam for optimal conformity to the curvature of the forehead.

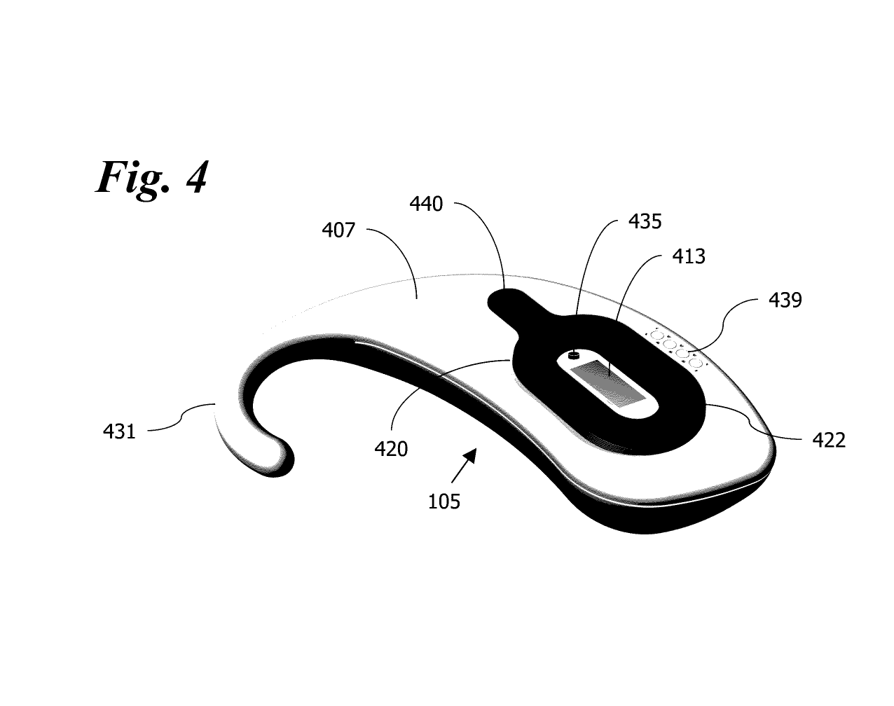

A more detailed view of an ear-worn version of the peripheral device 105 depicted in FIG. 1 is shown in FIG. 4. This version of the peripheral device is generally less stigmatizing than the forehead version 305. Herein, peripheral device 105 is shown with the skin-facing side up. It comprises a plastic injection molded enclosure 407 with a window 413 by which printed circuit board (PCB)-mounted LEDs and photodetector of the PPG sensor can be exposed to the skin. An adequate PPG sensor for use herein is model DCM03 reflectance pulse oximeter available from APMKorea, Daejeon, Korea (www.apmkr.com). A concave well 420 in the surface of the enclosure 407 provides for conformal fit to the curvature of the mastoid process area behind the ear, and also serves to block ambient light from interfering with the signal acquired at the photodetector. A double-sided disposable adhesive 422 is applied with each use of the device in concave well 420, and serves to hold the window 413 in stable contact with the skin, block ambient light by adhering to skin around the entire circumference of the window exposing the photodetector, and generally is capable of holding the entire peripheral device 105 in place behind the ear. A bendable hook 431 is optionally provided for further stabilization, though the primary support of the device 105 should be achieved by means of adhesive 422 for optimal signal quality. Skin temperature is obtained via thermally conductive nub 435, which is connected to a PCB-mounted thermistor. Conductive pads 439 provide means for recharging the enclosed battery of the device 105 when inserted into a matching charging stand. When the device is removed for recharging, the adhesive 422 can be peeled away from the device 105 and disposed of by pulling on non-adhesive tab 440.

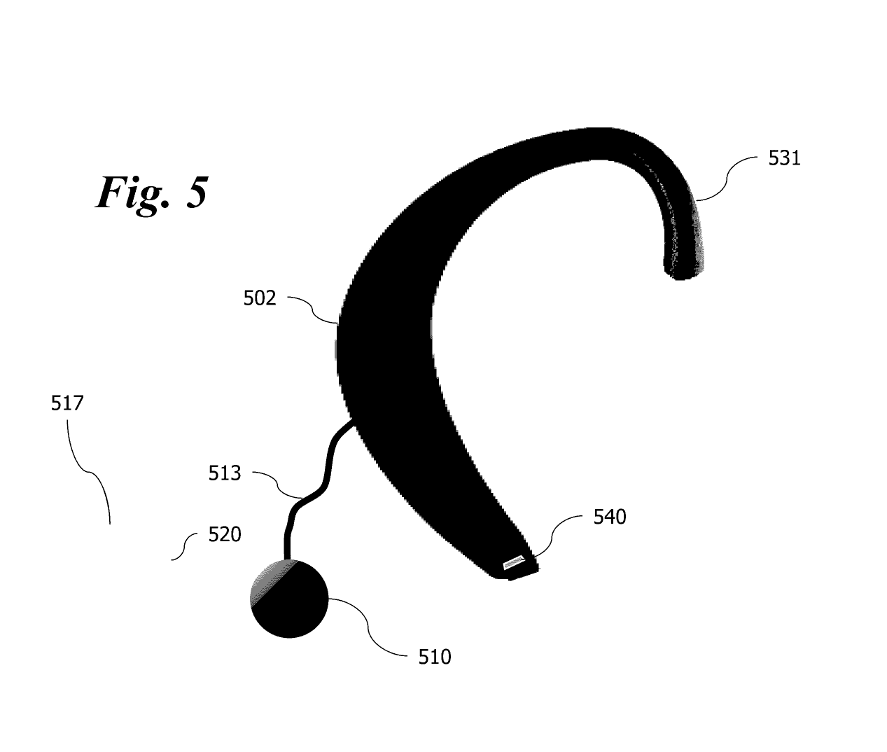

FIG. 5 shows an alternative embodiment of the ear-worn version of the peripheral device. In this embodiment, an enclosure 502 contains a battery and printed circuit board having thereon the microprocessor, A/D converters, firmware memory, data memory, radio and other hardware of the device. However, the LEDs and photodetector comprising the PPG sensor are located outside the enclosure 502 in a separate nodule 510, and connected to the circuitry in enclosure 502 via highly flexible cable 513. Nodule 510 may comprise a bead of baked ceramic encapsulating the PPG sensor; alternatively it may comprise an opaque flexible sponge-like material, as is used in conventional cabled forehead pulse oximeters used in hospitals; or it may comprise other durable materials capable of encapsulating the PPG sensor and being worn without irritation against the skin for long periods. In any case it should be opaque to infrared and optical wavelengths to eliminate ambient light. The cable 513 is well shielded, and comprises four wires facilitating ground, photodetector signal, and a power signal line for each of two LEDs. (One power line for just one LED can be used if only one PPG signal is desired; SpO2 will not be available in the case that only one LED is used). A disposable adhesive patch 517 is applied over the top of the nodule 510 to hold it in place against the skin over the mastoid process behind the ear. Adhesive patch 517 has a single-sided adhesive ring 520 around its circumference, but not in the center area 524 which covers the nodule 510. With nodule 510 held well in place by patch 517, the remaining enclosure 502 can be easily worn and supported over the ear by ear hook 531. Advantageously, this version of the ear-worn peripheral device decouples the motion of the comparatively heavy enclosure 502 from the PPG sensor, so that inertial motion of the battery, circuitry and enclosure from head motion of the patient tugs less on the PPG sensor, improving signal quality. Adhesive patch 517 is also easier to apply than the double-sided adhesive 42 shown in FIG. 4. Also, this version can be made to fit around hearing aids and glasses more easily than the version shown in FIG. 4. Disposable adhesive patch 517 can also be colored to match skin color, thereby improving wearability. A charging jack 540 may be provided as an alternative to conductive pads 435 from FIG. 4, and may accommodate a microUSB cable or the like.

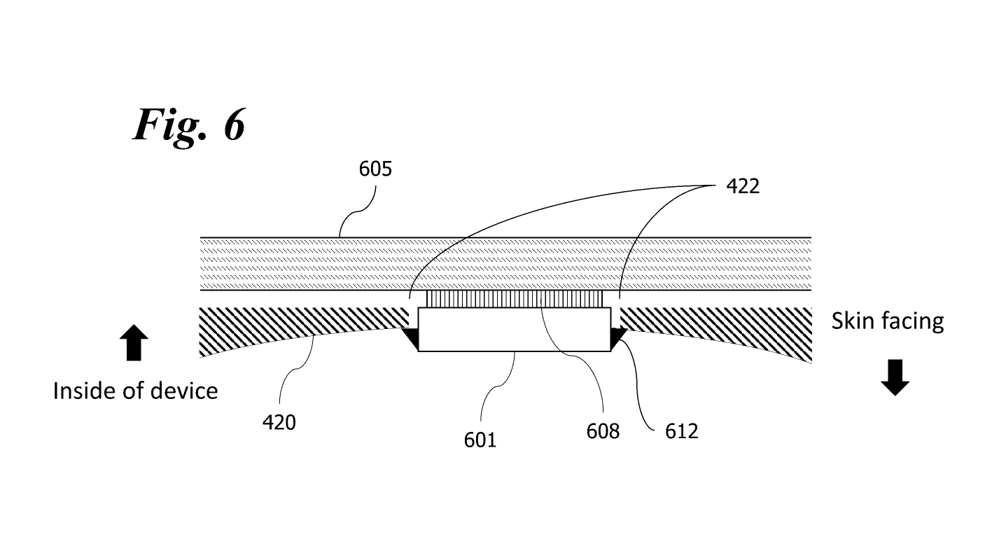

Turning to FIG. 6, a cross sectional view of the PPG sensor in window 422 of FIG. 4 is shown. Importantly, the PPG sensor 601 mounted on the multi-layer printed circuit board 605 of the device extends out of window 422 into concave well 420 approximately 1 millimeter by means of a riser 608, to ensure complete contact across the entirety of the PPG sensor surface with the skin. Importantly, no air gaps should exist between the PPG sensor surface and the skin. Around the edges of the PPG sensor 601, an opaque gasket or ring of opaque caulk 612 is applied to block all light from entering the sides of the sensor.

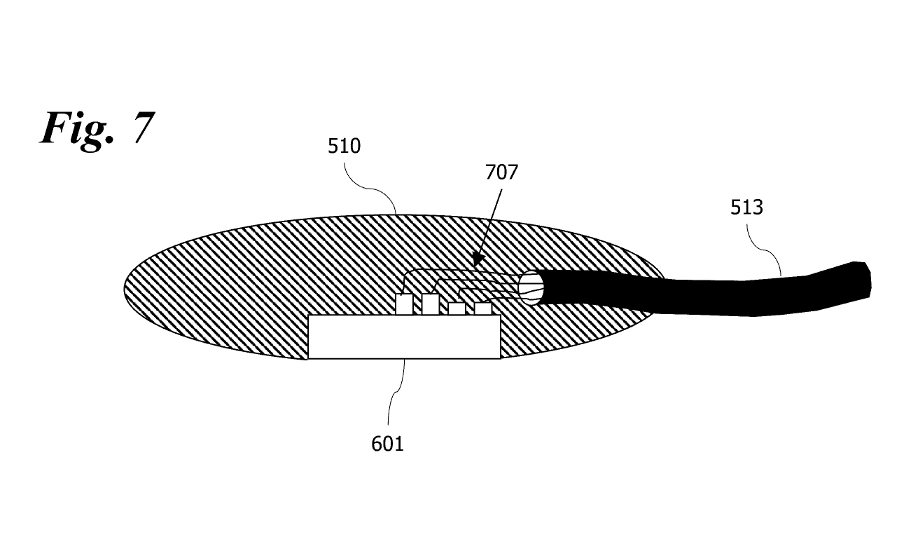

FIG. 7 similarly shows the cross sectional view of the PPG sensor 601 embedded in nodule 510 from FIG. 5. Nodule 510 is preferably convex on both surfaces, with the PPG sensor 601 located at the apex of convexity on the skin-facing side. This ensures maximum engagement of the PPG sensor with the skin, and mitigates any discomfort of sharp edges against the skin. Electrical leads 707 to the PPG sensor are shielded in cable 513, which is embedded in the matrix material of nodule 510.

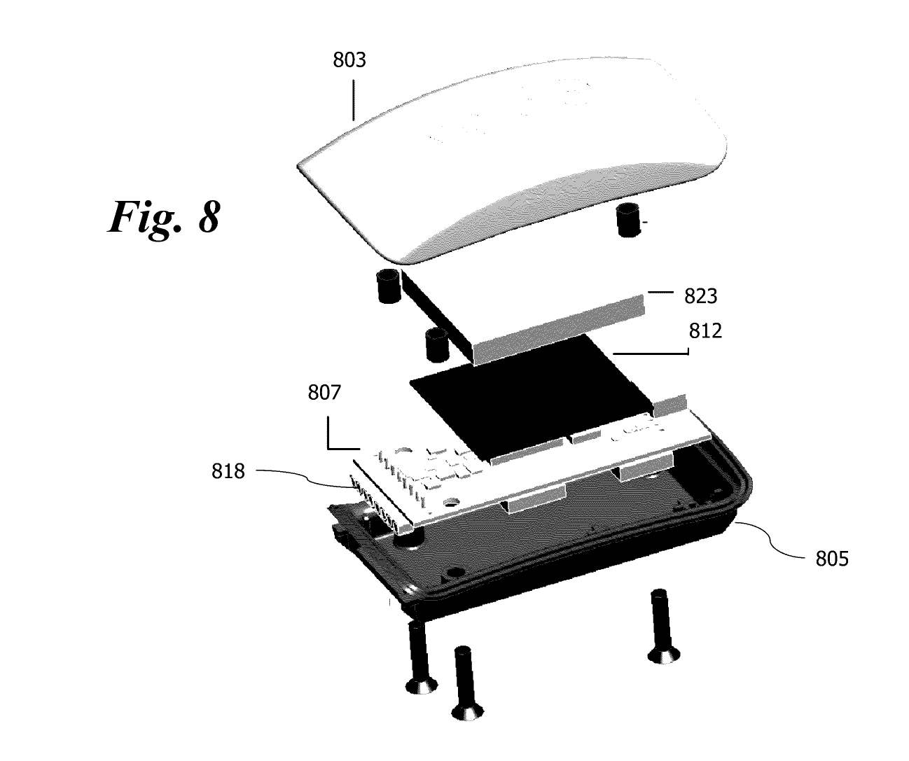

Turning to FIG. 8, an embodiment of the torso device of the present invention is shown to comprise a plastic (acrylonitrile butadiene styrene or ABS) injection molded upper shell 803 and lower shell 805 enclosure held together by screws. Enclosure plastics are medical grade. A circuit board 807 includes an MSP430 microprocessor from Texas Instruments, a radio for communications with the peripheral device, a Bluetooth standard radio for communication with a Bluetooth-enabled mobile phone, 3-axis accelerometer, bioimpedance integrated circuit, ECG integrated circuit, and other hardware. All external connections are concentrated in electrical contact pads 818 at one end of the device, and these receive contact with “pogo” type spring loaded conductive pins located in the chest belt harness 115 or adhesive harness 210. Power is provided by a 400 mAh lithium polymer rechargeable battery 823 which is separated from the PCB 807 by insulator 812.

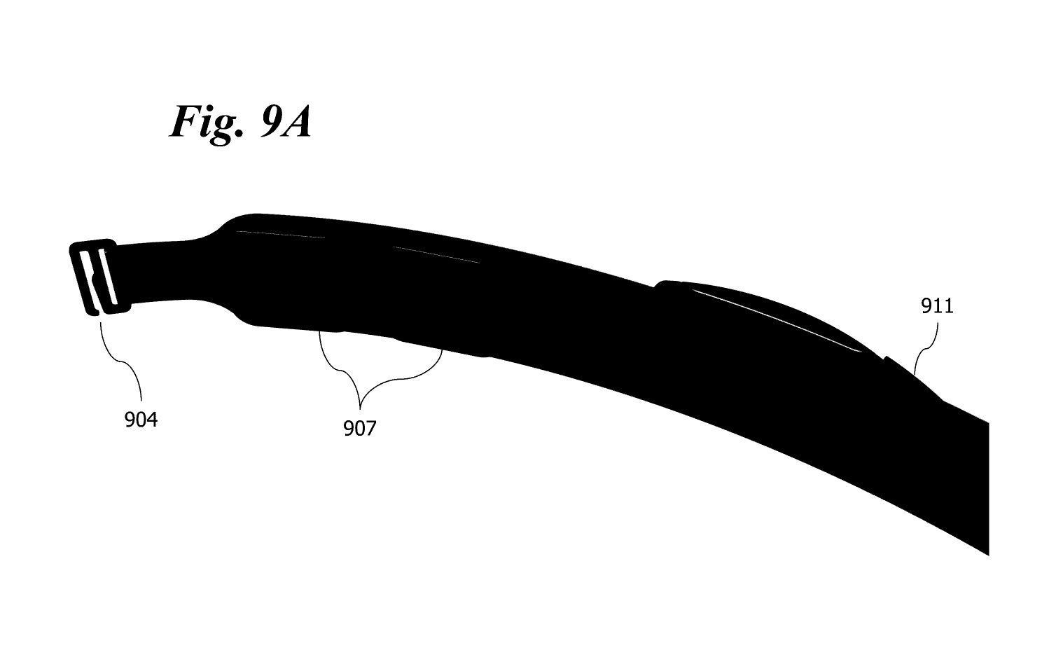

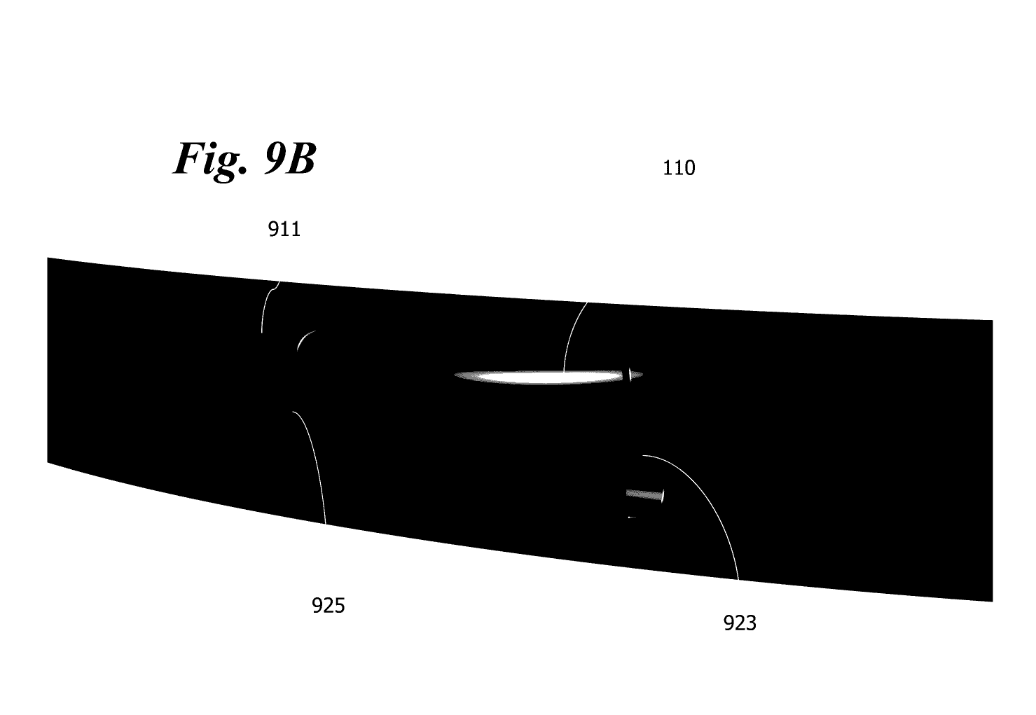

The chest belt harness 115 can be seen in detail in FIG. 9A to have a simple belt clasp 904 for easy connection with the belt loop, and four or more carbonized rubber dry electrodes 907 positioned facing the skin when the belt is worn. Electrical leads run inside a fabric tunnel of the belt to the harness 911 into which the torso device is snapped. FIG. 9B shows the outside front of the belt 115, where the torso device 110 has been snapped into place in the harness 911. A release cleft 923 is provided into which the patient can insert a finger to unsnap the torso device (for example to place it in a recharging stand). “Pogo” pin spring loaded connectors 925 located inside the cup of harness 911 contact the pads 818 of the torso device to make electrical connection with the dry electrodes. Spring-loaded ball bearing protrusion located at the opposite end of the harness 911 fit into corresponding indentations in the torso device enclosure to achieve a snap fit.

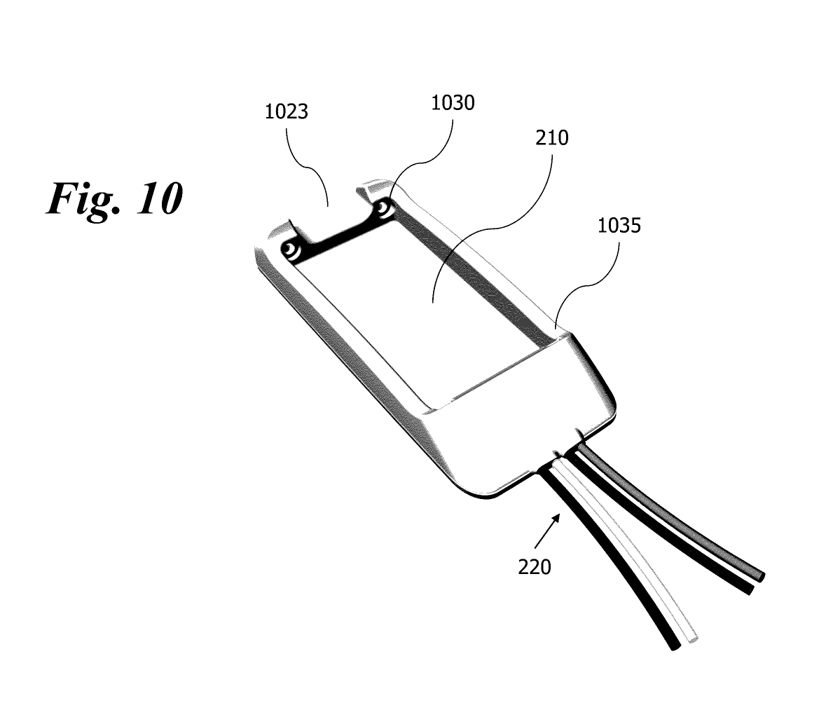

The adhesive harness 210 can be seen in detail in FIG. 10 to similarly have spring loaded ball bearing protrusions 1030 for securing the torso device as with the chest harness, and a release cleft 1023 for easy removal of the device from the harness with the finger. Spring loaded electrical contact pins at 1035 interface with the pads 818 of the torso device to provide electrical connection via leads 220 to at least four skin adhesive electrodes. The wires can be color coded to help the patient attach the leads to the correct electrode locations.

An important aspect of the present invention is the capability to independently measure with distinct wireless devices biosignals that must be accurately combined to produce new vital sign features. In particular, the determination of pulse transit time, diastolic relaxation time and pulse pressure index depend on accurate time differentials between landmarks on the ECG signal obtained from the torso device and landmarks on the PPG signal from the peripheral device. Synchronizing biosignals across devices that do not share a common electrical connection can be challenging: Even the most accurate of onboard oscillator crystals used for time counters or onboard clocks can drift, especially with differences in temperature; lost radio packets need to be accounted for to avoid the signals getting out of step; devices must be resynchronized with each power cycle or battery discharge. At the same time, the need to minimize battery size in order to keep device size small imposes a constraint on unfettered use of device radios for hyper-frequent synchronization of onboard timers. Radio drop-outs also poses the risk of lost data, which can negatively impact signal processing to find landmarks in the biosignal wave form.

In order to meet these challenges, in a preferred embodiment the peripheral device maintains a circular buffer of data packets, each packet comprising a predetermined number of samples of biosignal data. The torso device also maintains a “receive” buffer of packets it receives from the peripheral device, from which it works to combine data with biosignals it has collected from its sensors. Samples are grouped into packets in order to cut down on the time that the radio must be turned on to transmit, since the radio can efficiently transmit large packets of many samples much faster than the actual sampling rate of the biosignal. Thus, the radio can send a large packet of data and then be placed into an energy conserving mode until the next packet needs to be sent.

Upon peripheral device power-up, the circular buffer is first filled with a predetermined number of packets, and only after this packet count is attained is the radio first turned on to send all these packets to the torso device at once. The receipt of each packet sent must generally be acknowledged by the torso device, and only then will be removed from the circular buffer of the peripheral device. This initial burst of packets serves to fill the “receive” buffer of the torso device. This provides a backlog of packets which the torso device can consume in the event that further transmissions of packets from the peripheral device to the torso device temporarily fail and must be retried, due to ambient interference and noise. After sending a burst of the predetermined number of packets to fill the “receive” buffer of the torso device, the peripheral device thereafter sends packets as they become available, and its circular buffer of packets generally remains near-empty in the absence of radio transmission failures.

As mentioned, each packet sent from the peripheral device to the torso device must be acknowledged as received by the torso device to the peripheral device, typically by means of a brief acknowledgement reply which can preferably also include a packet identifier. If the acknowledgement is not received within a specified time window, the peripheral device assumes the transmission failed, and it resends the packet. Given the slower sampling rate of the biosignal data as compared to the rapid speed of data transmission, this resend can occur a number of times before acquisition of enough samples to form the next new packet, providing some latency for catching up with transmission without true data loss. Moreover, the peripheral device circular buffer provides a FIFO temporary store for acquired biosignal data in the event that radio communications to the torso device fail for longer due to transient noise and ambient interference; data can be inventoried without loss, up to the maximum size of the circular buffer. If radio transmissions continue to fail as the circular buffer is refilled to capacity, additional packets are eliminated on a first-in, first-out (FIFO) basis, and only then is biosignal data truly lost. When radio communication is next reestablished with the torso device, the backlog of packets inventoried in the circular buffer is transmitted in a burst to refill the “receive” buffer of the torso device, much like at power-up.

The sampling rates of the biosignals acquired by the peripheral device stand in some known ratio to the sampling rates of biosignals acquired by the torso device; in the preferred embodiment at least one biosignal from the peripheral device is sampled at the same rate as a biosignal from the torso device, so that sample counts can be directly compared as a means of synchronizing those signals and all other signals in relation to their respective sampling rates. By way of example, the PPG signals can be acquired at 250 Hz by the peripheral device, and the BIOZ or ECG biosignal can be acquired at 250 Hz by the torso device, so that sample tallies of each provide a baseline time synchronization of the biosignals for calculation of time differential features and other timestamps of the data. Generally, if the sampling rates are different, the ratio of rates can be used to determine how many samples of one biosignal correspond to samples of another biosignal to preserve synchronization information.

However, loss of data in radio transmission can cause a failure of this count-based synchronization. Therefore, a packet count of received packets is maintained on the torso device. It is known what the predetermined number of packets is that triggers the initial filling of the “receive” buffer, and accounting of packets is made from this baseline. Biosignal data acquired by the torso device is also tallied in parallel in “packets” of the same number of samples (or a known ratio of samples if the sampling rates are set to different frequencies); if the “receive” buffer of the torso device is depleted, and the number of samples obtained from a reference biosignal acquired by the torso device reaches a quantity equating to a “packet” of data from the peripheral device, it is assumed the peripheral device packet is permanently lost, and the data time series corresponding to peripheral device-acquired biosignals that the torso device is combining with its own biosignals for transmission to the mobile phone is filled with a “packet” of null, zero or other value designated as an indicator of lost data. In this way, the synchronization of the biosignals is preserved in relation to their respective sampling rates (typically the same sampling rate), since there is no timestamp associated with the data packets sent by the peripheral device. Filling in with replacement samples maintains the sequential alignment.

Generally, therefore, the circular buffer of the peripheral device is kept near zero packets, after the initial burst of packets on power-up fills the torso device “receive” buffer. As each batch of samples comprising a packet is formed on the peripheral device, several attempts are made to transmit this packet to the torso device. In the event that sustained radio interference prevents successful sending of the packet before a second packet of samples accumulates on the peripheral device, the circular buffer will begin to backlog the packets, and the torso device “receive” buffer will similarly begin to consume its backlog of packets filled with the initial burst. At any time prior to the filling to capacity of the circular buffer on the peripheral device, if radio transmissions are successfully reestablished, all pending packets are sent to the torso device, effectively refilling the “receive” buffer and emptying the circular buffer. Only when radio transmissions fail for an extended period, and the circular buffer reaches capacity at the same time as the torso device depletes all packets in its “receive” buffer, are packets of data lost. This will occur on the peripheral device by simple elimination of the packets on a FIFO basis. A commensurate data gap will be filled in the data stream being assembled by the torso device for transmission to the mobile phone by insertion of a full packet of nulls or zeros. The sample counts at both sides of the wireless communication are thus kept in synch.

Another problem with synchronizing sample counts however arises as mentioned above due to subtle differences in crystal frequency in each device. Though nominally set to the same frequency to drive biosignal sampling, differences in clock speeds due to manufacturing tolerances as well as differences that arise randomly in oscillator performance due to temperature differentials, can give rise to effectively different samples counts in the same true window of time. In one approach to this issue, a count of samples is maintained by the torso device of both the biosignals received from the peripheral device and from biosignals acquired by the torso device, and at specified intervals, if there is a discrepancy in the count in relation to expected sample numbers based on sampling rates, then excess samples are eliminated from one or the other sample stream. However, a much simpler better and more tractable approach is to actually set the clock speed of one of the devices to be slightly higher than the other. In this way, the device with the lower clock speed provides the “true” clock tick and the higher clock speed device data samples are forced to fall on the “true” ticks by intermittently removing the most recent sample when the total number of samples acquired is at least one sample greater than the lower clock speed device's total number of acquired samples. Doing this keeps the sampling consistent with a single clock and sample adjustments only need to be made on the samples from the higher clock speed device.

The mobile phone of the present invention preferably has a high resolution display and sufficient onboard processing power to render real-time biosignal data for review by the patient or a clinician on-screen. Mobile phones based on the Android operating system, Windows Phone operating system and Apple iOS operating system are quite adequate to be used in the present invention.

Turning now to the process for multivariate analysis of data collected by the inventive device, a number of different kernel-based multivariate estimator methods may be used for analysis on the remote computer platform of the uploaded data. According to this approach, a set of vital sign features are observed at a given moment in time to form a multidimensional “observation” (vector). Successive observations of the vital sign features form a multivariate time series of these vectors. An empirical model is generated as described below from exemplary observations of vital sign features collected in baseline or normal health (and indeed can be learned from the instant patient to form a personalized model). The model, once trained, can be used to generate multivariate estimates of the expected values of the vital sign features, when presented with an input of a new observation of the features. Differences between the estimates and the monitored observations form the basis for a determination of health status. Advantageously, the collective use of multiple vital signs together effectively informs the model's estimate of each feature—in essence, the model learns the way that the vital signs interrelate.

What is generally intended by the term “kernel-based” is a multivariate estimator that operates with a library of exemplary observations (the learned data) on an input observation using a kernel function for comparisons. A kernel function suitable for this multivariate analysis according to the invention generally yields a scalar value (a “similarity”) on a comparison of the input observation to an exemplary observation from the library. The scalar similarity can then be used in generating an estimate as a weighted sum of at least some of the exemplars. For example, using Nadaraya-Watson kernel regression, the kernel function is used to generate estimates according to:

$$\begin{matrix}Inferential form \text{:} {y}_{est} = \frac{{\sum }_{i = 1}^{L} {y}_{i}^{out} K ( {x}_{new} , {x}_{i}^{in} )}{{\sum }_{i = 1}^{L} K ( {x}_{new} , {x}_{i}^{in} )} & ( 1 ) \\ Autoassociative form \text{:} {x}_{est} = \frac{{\sum }_{i = 1}^{L} {x}_{i} K ( {x}_{new} , {x}_{i} )}{{\sum }_{i = 1}^{L} K ( {x}_{new} , {x}_{i} )} & ( 2 )\end{matrix}$$

where X_{new} is the input multivariate observation of physiological features, Xᵢ are the exemplary multivariate observations of physiological features, Xₑₛₜ are the estimated multivariate observations, and K is the kernel function. In the inferential case, exemplars comprise a portion Xᵢ comprising some of the physiological features, and a portion Yᵢ comprising the remaining features, X_{new} has just the features in Xᵢ, and Yₑₛₜ is the inferential estimate of those Yᵢ features. In the autoassociative case, all features are included in X_{new}, Xᵢ and in the Xₑₛₜ together—all estimates are also in the input.

The kernel function, by one approach, provides a similarity scalar result for the comparison of two identically-dimensioned observations, which:
1. Lies in a scalar range, the range being bounded at each end;
2. Has a value of one of the bounded ends, if the two vectors are identical;
3. Changes monotonically over the scalar range; and
4. Has an absolute value that increases as the two vectors approach being identical.

In one example, kernel functions may be selected from the following forms:

$$\begin{matrix}{K}_{h} ( {x}_{a} , {x}_{b} ) = {e}^{- \frac{{‖ {x}_{a} - {x}_{b} ‖}^{2}}{h}} & ( 3 ) \\ {K}_{h} ( {x}_{a} , {x}_{b} ) = {( 1 + \frac{{‖ {x}_{a} - {x}_{b} ‖}^{λ}}{h} )}^{- 1} & ( 4 ) \\ {K}_{h} ( {x}_{a} , {x}_{b} ) = 1 - \frac{{‖ {x}_{a} - {x}_{b} ‖}^{λ}}{h} & ( 5 )\end{matrix}$$

where Xₐ and X_{b} are input observations (vectors). The vector difference, or “norm”, of the two vectors is used; generally this is the 2-norm, but could also be the 1-norm or p-norm. The parameter h is generally a constant that is often called the “bandwidth” of the kernel, and affects the size of the “field” over which each exemplar returns a significant result. The power λ may also be used, but can be set equal to one. It is possible to employ a different h and λ for each exemplar Xᵢ. Preferably, when using kernels employing the vector difference or norm, the measured data should first be normalized to a range of 0 to 1 (or other selected range), e.g., by adding to or subtracting from all sensor values the value of the minimum reading of that sensor data set, and then dividing all results by the range for that sensor; or normalized by converting the data to zero-centered mean data with a standard deviation set to one (or some other constant). Furthermore, a kernel function according to the invention can also be defined in terms of the elements of the observations, that is, a similarity is determined in each dimension of the vectors, and those individual elemental similarities are combined in some fashion to provide an overall vector similarity. Typically, this may be as simple as averaging the elemental similarities for the kernel comparison of any two vectors x and y:

$$K ( x , y ) = \frac{1}{L} {\sum }_{m = 1}^{L} K ( {x}_{m} , {y}_{m} ) \qquad ( 6 )$$

Then, elemental kernel functions that may be used according to the invention include, without limitation:

$$\begin{matrix}{K}_{h} ( {x}_{m} , {y}_{m} ) = {e}^{- \frac{{\vert {x}_{m} - {y}_{m} \vert}^{2}}{h}} & ( 7 ) \\ {K}_{h} ( {x}_{m} , {y}_{m} ) = {( 1 + \frac{{\vert {x}_{m} - {y}_{m} \vert}^{λ}}{h} )}^{- 1} & ( 8 ) \\ {K}_{h} ( {x}_{m} , {y}_{m} ) = 1 - \frac{{\vert {x}_{m} - {y}_{m} \vert}^{λ}}{h} & ( 9 )\end{matrix}$$

The bandwidth h may be selected in the case of elemental kernels such as those shown above, to be some kind of measure of the expected range of the m^{th} parameter of the observation vectors. This could be determined, for example, by finding the difference between the maximum value and minimum value of a parameter across all exemplars. Alternatively, it can be set using domain knowledge irrespective of the data present in the exemplars or reference vectors, e.g., by setting the expected range of a heart rate parameter to be 40 to 180 beats per second on the basis of reasonable physiological expectation, and thus h equals “140” for the m^{th} parameter in the model which is the heart rate.

Similarity-Based Modeling may be used as the kernel-based multivariate estimator. Three types of SBM models can be used for human data analysis tasks: 1) a fixed SBM model, 2) a localized SBM model that localizes using a bounding constraint, and 3) a localized SBM model that localizes using a nearest neighbor approach. The fixed SBM modeling approach generates estimates using the equation below.

$${\hat{x}}_{in} ( t ) = \frac{{D ( {D}^{T} ⊗ D )}^{- 1} ( {D}^{T} ⊗ {x}_{in} ( t ) )}{\sum {( {D}^{T} ⊗ D )}^{- 1} ( {D}^{T} ⊗ {x}_{in} ( t ) )} \qquad ( 10 )$$

Here, D is a static m-by-n matrix of data consisting of n training data vectors with m physiological features, pre-selected from normal data during a training phase. The kernel function K is present as a kernel operator [symbol] whereby each column vector from the first operand (which can be a matrix, such as D is) is compared using one of the kernel functions described above, to each row vector of the second operand (which can also be a matrix). The monitored input observation is here shown as xᵢₙ(t), and the autoassociative estimate is shown as \hat{x}ᵢₙ(t). In contrast, localized SBM (LSBM) is given by the following equation:

$${\hat{x}}_{in} ( t ) = \frac{D ( t ) {( {D ( t )}^{T} ⊗ D ( t ) )}^{- 1} ( {D ( t )}^{T} ⊗ {x}_{in} ( t ) )}{\sum {( {D ( t )}^{T} ⊗ D ( t ) )}^{- 1} ( {D ( t )}^{T} ⊗ {x}_{in} ( t ) )} , \\ D ( t ) = \{ H \vert F ( H , {x}_{in} ( t ) ) \} \qquad ( 11 )$$

Although similar in form to the fixed SBM model, here the D matrix is redefined at each step in time using a localizing function F(·) based on the current input vector xᵢₙ(t) and a normal data reference matrix H. Accordingly, matrix H contains a large set of exemplars of normal data observations, and function F selects a smaller set D using each input observation. By way of example, F can utilize a “nearest neighbor” approach to identify a set of exemplars to constitute D for the current observation as those exemplars that fall within a neighborhood of the input observation in m-dimensional space, where m is the number of features. As another example, function F can compare the input observation to the exemplars for similarity using a kernel-based comparison, and select a preselected fraction of the most similar exemplars to constitute D. Other methods of localization are contemplated by the invention, including selection on the basis of fewer than all of the physiological features, and also selection on the basis of a distinct parameter not among the features, but associated with each exemplar, such as an ambient condition measure.

One method of residual testing that may be employed in the analytical aspect of the monitoring platform disclosed herein is a multivariate density estimation approach can be applied to the residual data. This has the effect of fusing the residuals from multiple vital sign features for which estimates are made with the model, into a single actionable index of physiological change that can be used to evaluate overall priority for medical care. The approximated densities in the normal behavior of the data are used to determine the likelihood (in the form of a multivariate health index (MHI)) that a new data point is part of the normal behavior distribution. The density estimates are calculated using a non-parametric kernel estimator with a Gaussian kernel. The estimator is shown in the equation below. The resulting density function is essentially a mixture of N individual multivariate Gaussian functions each centered at xᵢ:

$$\hat{f} ( x ) = \frac{1}{{N ( 2 π )}^{d / 2} {h}^{d}} {\sum }_{i = 1}^{N} exp [ - \frac{1}{2} \frac{‖ x - {x}_{i} ‖}{{h}^{2}} ] \qquad ( 12 )$$

where N is the number of training vectors, h is a bandwidth parameter, d is the dimensionality of the vectors, and \hat{f}(x) is a scalar likelihood. Importantly, the X and Xᵢ here are not multivariate observations of physiological features, but are instead multivariate residual observations derived from the original observations by differencing with the estimates. Importantly also, the density “estimation” here is not the same as the estimation process described above for estimating physiological feature values based on measured values; the “estimate” here is empirically mapping out a probability distribution for residuals using the normal multivariate residual exemplars, as a Gaussian mixture model. This estimated distribution is then used to compute a likelihood that a new multivariate residual from an input observation of physiological features is a member of that distribution or not. The exemplars Xᵢ can be selected from regions of normal data residuals generated by SBM using test data that is deemed “normal” or representative of desired or stable physiological behavior. Before the density estimates are made, all residuals are scaled to have unit variance and zero mean, or at least are scaled to have unit variance. The means and standard deviations used for the scaling procedure are calculated from known normal data residuals.

Analytical results are presented to a medical clinician preferably by means of a secure web page in which time series of MHI can be evaluated to ascertain stability of health in an at-home patient. By means of the invention described hereinabove, high fidelity continuous multivariate physiological data is automatically collected, uploaded, analyzed and processed to inform medical practitioners of subtle early warning signs of incipient health degradation, so that early, easy, low-cost steps can be taken to mitigate the patient's health issue and keep the patient from being eventually hospitalized.

What is claimed is:

1. A system for monitoring human health comprising:
a wearable torso device comprising a first firmware-programmed microprocessor, a first internal memory, and at least one analog-to-digital converter, disposed to continuously measure at least an electrocardiographic signal from at least two electrodes;
a peripheral device in wireless connectivity with said torso device, comprising a second firmware-programmed microprocessor, a second internal memory, and having at least one light source and a light sensitive element arranged with respect to said light source for quantitatively measuring light from said light source that has passed through subcutaneous tissue when said peripheral device is positioned on skin, thereby providing a photoplethysmographic signal that is communicated to the torso device wirelessly; wherein by means of automatic execution of firmware in said first and second microprocessors
said peripheral device is disposed to accumulate data packets of photoplethysmographic signal in a first circular buffer in said first internal memory and periodically transmit at least one data packet to said torso device and upon acknowledgement that the transmitted packet was received by said torso device, to remove the transmitted packet from said first circular buffer;
said torso device is disposed to store said data packets received from said peripheral device in a second circular buffer in said second internal memory, maintain a count of packets received, and remove packets from said second circular buffer upon processing each packet with matched electrocardiographic signal data; and when the wireless connectivity is interrupted for an extended period
said peripheral device is disposed to overwrite an unsent packet with a new packet on a first-in-first-out basis when said first circular buffer is full while said torso device is disposed to generate a packet of null data for matching and processing with electrocardiographic signal data and to increment said packet counter, when said second circular buffer is empty, in order to maintain synchronization of said photoplethysmographic signal with said electrocardiographic signal.

2. The system according to claim 1, further comprising a mobile phone disposed to receive wireless transmissions of data from said torso device inclusive of data from said peripheral device.

3. The system according to claim 1 further comprising a headband disposed to hold the peripheral device against a forehead, and said peripheral device comprises an enclosure that is curved to conform to the curvature of the human forehead.

4. The system according to claim 1 wherein said peripheral device further comprises an enclosure with a concave well disposed to be attached against skin over the mastoid process of a wearer with a double-sided adhesive.

5. The system according to claim 1 wherein said peripheral device further comprises
an enclosure containing said first microprocessor and said first internal memory;
a sensor nodule containing said light source and said light sensitive element, connected to said enclosure by a flexible electrical cable; and
an opaque adhesive patch adapted to cover said nodule and adhere to skin circumferentially around said nodule.

6. The system according to claim 5 wherein said peripheral enclosure is hook-shaped so as to be adapted to hang over the ear of a wearer.

7. The system according to claim 1 wherein:
said peripheral device further comprises a first oscillator clock at a first frequency;
said torso device further comprises a second oscillator clock at a second frequency selected to be intentionally and slightly different from said first frequency such that the count of samples of electrocardiographic signal collected by said torso device differs by one sample from the count of samples of photoplethysmographic signal collected by said peripheral device after a selected period of time;
said second microprocessor under execution of firmware eliminates a sample from the one of the set of the electrocardiographic signal and the photoplethysmographic signal that has an additional sample, in order to deterministically correct for clock drift between said first and second clocks.

8. A system for monitoring human health comprising:
a wearable torso device comprising a first firmware-programmed microprocessor, a first internal memory, a first radio and at least one analog-to-digital converter, disposed to measure at least an electrocardiographic signal from at least two electrodes;
a peripheral device comprising
an enclosure containing a second firmware-programmed microprocessor, a second internal memory, and a second radio;
a sensor nodule connected to said enclosure by a flexible electrical cable and containing at least one light source and a light sensitive element arranged with respect to said light source for quantitatively measuring light from said light source that has passed through subcutaneous tissue when said sensor nodule is positioned on skin, to measure a photoplethysmographic signal; and
an opaque adhesive patch adapted to cover said nodule and adhere to skin circumferentially around said nodule;
and
a mobile phone disposed to receive via wireless transmission said electrocardiographic signal and said photoplethysmographic signal.

9. The system according to claim 8, wherein said peripheral device is configured to wirelessly transmit said photoplethysmographic signal to said torso device, and said torso device is configured to transmit the combination of said photoplethysmographic signal and said electrocardiographic signal to said mobile phone.

10. The system according to claim 9, wherein:
said peripheral device further comprises a first oscillator clock at a first frequency;
said torso device further comprises a second oscillator clock at a second frequency selected to be intentionally and slightly slower than said first frequency such that the count of samples of electrocardiographic signal collected by said torso device is less by one sample from the count of samples of photoplethysmographic signal collected by said peripheral device after a selected period of time;
said second microprocessor under execution of firmware eliminates a sample from the photoplethysmographic signal, in order to deterministically correct for clock drift between said first and second clocks.

11. A method for monitoring a physiological status of a human:
acquiring a photoplethysmographic signal from the mastoid process of the human using a wearable PPG sensor;
acquiring an electrocardiographic signal from the torso of the human using a wearable ECG sensor;
wirelessly receiving said photoplethysmographic signal and said electrocardiographic signal in a mobile phone;
uploading said signals from said mobile phone to a remote analysis server;
determining in said remote analysis server time differences between a first repeating landmark in said electrocardiographic signal and a subsequent second repeating landmark in said photoplethysmographic signal to provide a time series of time differences; and
assessing in said remote analysis server a physiological status of the human based on a multivariate residual-based model that uses said time differences as a variable.

12. The method according to claim 11 wherein said first landmark is a QRS complex of said electrocardiographic signal and said second landmark is an inflexion point of said photoplethysmographic signal indicative of blood pulse arrival, to yield time differences indicative of a pulse transit time.

13. The method according to claim 11 wherein said first landmark is a QRS complex of said electrocardiographic signal and said second landmark is an inflexion point of said photoplethysmographic signal from decreasing light transmission to increasing light transmission, yielding time differences indicative of a diastolic relaxation rate.

14. The method according to claim 11, wherein said wearable PPG sensor has a microprocessor clock speed that is slightly different from the a microprocessor clock speed of said wearable ECG sensor, such that the count of samples of electrocardiographic signal collected by said wearable ECG sensor differs by one sample from the count of samples of photoplethysmographic signal collected by said wearable PPG sensor after a selected period of time, and further comprising the step of eliminating a sample from the one of the set of the electrocardiographic signal and the photoplethysmographic signal that has an additional sample, in order to deterministically correct for clock drift between said ECG sensor and said PPG sensor.
